(19) United States
(12) Patent Application Publication
(10) Pub. No.: US 2018/0362957 A1
(43) Pub. Date: Dec. 20, 2018

(54) RuBisCO Protein-Based Films

(71) Applicant: R. J. Reynolds Tobacco Company, Winston-Salem, NC (US)

(72) Inventors: Andries Sebastian, Winston-Salem, NC (US); John P. Mua, Winston-Salem, NC (US); Neil McClanahan, Winston-Salem, NC (US); Sammy Eni Eni, Winston-Salem, NC (US); Thaddeus J. Jackson, Winston-Salem, NC (US); Savannah Johnson, Winston-Salem, NC (US)

(21) Appl. No.: 15/623,006

(22) Filed: Jun. 14, 2017

(51) Int. Cl. C12N 9/88; B29C 39/38; B29C 39/36; B29B 7/00

(52) U.S. Cl. CPC C12N 9/88; C12Y 401/01039; B29L 2007/00; B29C 39/36; B29B 7/005; B29C 39/38

(57) ABSTRACT

Ribulose-1,5-bisphosphate oxygenase (RuBisCO) protein films and a method of producing RuBisCO films are disclosed herein. A method of producing one or more RuBisCO protein films includes obtaining RubBisCO, for example from tobacco, combining the RuBisCO with one or more solvents, where the one or more solvents may be about 10% w/v the RuBisCO, mixing the RuBisCO and the one or more solvents to form a slurry, heating the slurry to about 70 degrees C., cooling the slurry to at least about 45 degrees C., dispensing the slurry into one or more molds for film formation, drying the slurry in the one more molds, and removing the one or more RuBisCO protein films formed within the one or more molds.

### BACKGROUND

Numerous uses of tobacco and tobacco-based products have been proposed. For example, tobacco has been smoked in pipes, cigarettes, and cigars. See e.g. Tobacco Production, Chemistry and Technology, Davis et al. (Eds.) p. 346 (1999). More recently, there has been focus on various ways of providing various sensations of smoking, without delivering to a smoker quantities of incomplete combustion and pyrolysis products that may result from the burning of tobacco. See e.g., the background art set forth in U.S. Pat. No. 7,503,330 to Borschke et al. and U.S. Pat. No. 7,726,320 to Robinson et al., U.S. Pat. Pub. No. 2014/0261495 to Novak, III et al., and U.S. Pat. Pub. No. 2014/0096780 to Gerardi. In addition to smoking, tobacco may also be used in so-called smokeless forms. See e.g. the background art set forth in U.S. Pat. Pub. No. 2012/0272976 to Byrd et al. Furthermore, various materials derived and/or extracted from tobacco have been proposed to have uses in certain industrial applications. See e.g. U.S. Pat. No. 2,098,836 to Ressler, U.S. Pat. No. 2,232,662 to Hockenyos, U.S. Pat. No. 4,347,324 to Wildman et al., U.S. Pat. No. 4,289,147 to Wildman et al., U.S. Pat. Pub. Nos. 2011/01287681 to DeVall, and 2012/0260929 to Coleman et al.

Methods of extracting proteins from tobacco and tobacco components have been proposed in U.S. Pat. No. 9,301,544 to Mua et al., U.S. Pat. No. 9,175,052 to Gerardi et al., U.S. Pat. Pub. No. 2016/0192697 to Mua et al., and U.S. Pat. Pub. No. 2016/0029663 to Gerardi et al. It may be desirable to utilize protein compositions extracted from tobacco for various purposes, such as the production of protein-based films, including, but not limited to, edible protein films for use in biomedical, pharmaceutical, or food industry applications. Protein-based films are known in the art, including those sourced from collagen, gelatin, corn zein, wheat gluten, soy protein, casein, mung bean protein, and the like.

Generally, protein films may have desirable oxygen barrier properties, but may not have desirable water vapor properties. The properties protein films exhibit may be dependent on the association of protein chains through various types of bonding, for example through hydrogen, ionic, hydrophobic, and/or covalent bonding. This association of protein chains with other protein chains may produce films, and these films may be affected by the nature and distribution of various residues, such as polar residues, hydrophobic residues, and amino acid residues. Conventionally, polymer chain-to-chain interactions have resulted in stronger films, but these films tend to be less permeable to gases, vapor, or liquids. For example, polymers containing groups that utilize hydrogen or ionic bonding have conventionally resulted in films capable of functioning as oxygen barriers, but also in films that may demonstrate susceptibility to moisture. Alternatively, polymers with a large number of hydrophobic groups may not function well as oxygen barriers, but may function well as moisture barriers.

Conventional protein films may utilize protein obtained from a variety of sources. However, these protein sources (e.g. animal sources) may not be abundant and may limited in availability and/or be expensive to obtain. Sourcing protein for films from more abundant sources such as soy and/or milk casein have been proposed, but these sources are also traditional food sources. It may be desirable to utilize other, non-food protein sources for the production of protein films, including but not limited to, tobacco, Kudzu, alfalfa, switchgrass, hemp, or any other grasses or shrubs. It may be further desirable for the resulting protein films to function as an oxygen and/or moisture barrier.

Ribulose-1,5-bisphosphate carboxylase/oxygenase (hereinafter “RuBisCO”) is considered the most abundant plant protein known, as it is an enzyme involved in the first major step carbon fixation by plants and other photosynthetic organisms, making it an abundant, potentially non-food, protein source that may be desirable in the production of protein films. For example, RuBisCO may comprise up to about 25% of the total protein content of a leaf and up to about 10% of the solid matter of a leaf. Furthermore, tobacco plants may have the highest potential yield per acre of RuBisCO of all plants, without the limitation of also being a traditional food source.

### SUMMARY

The present disclosure is directed to inventive ribulose-1,5-bisphosphate oxygenase (RuBisCO) protein films and methods of producing them. In one aspect, a method of producing one or more RuBisCO protein films may include the steps of: obtaining RuBisCO protein; combining the RuBisCO protein with one or more solvents, where the one or more solvents may be 10% w/v the RuBisCO protein; mixing the RuBisCO and the one or more solvents to form a slurry; heating the slurry to about 70 degrees C.; cooling the slurry to at least about 45 degrees C.; dispensing the slurry into one or more molds for film formation; drying the slurry in the one more molds; and, removing the one or more RuBisCO protein films formed within the one or more molds.

In some embodiments, the RuBisCO protein may be obtained from one or more tobacco plants. In other embodiments, the method further may comprise purifying the obtained RuBisCO protein. In still other embodiments, the one or more solvent solutions may be selected from a group consisting of: water, ethanol, glycerol, propylene glycol, polypropylene glycol, hexane, a citrate solution, a phosphate solution, a chloride solution, a sodium sulfate solution, a potassium sulfate solution, a sodium hydroxide solution, a potassium hydroxide solution, a calcium hydroxide solution, a magnesium hydroxide solution, a hydrochloric acid solution, a phosphoric acid solution, a citric acid solution, a sodium carbonate solution, and a potassium carbonate solution.

In some embodiments, the method may further comprise adding one or more additional additives selected from a group consisting of: one or more crosslinking agents, one or more plasticizers, and one or more reinforcers.

In some embodiments, the crosslinking agent(s) may be selected from a group consisting of glutaraldehyde, glyoxal, and formaldehyde. In some embodiments, the plasticizer(s) may be selected from a group consisting of monosaccharides, disaccharides, oligosaccharides, polyols, and lipids. In some embodiments, the reinforcers may be selected from a group consisting of sodium alginate, pectin, carrageenan, gellan, agar, gum acacia or gum Arabic, tragacanth, karaya, guar, locust bean, pullulan, xanthan, hydroxypropyl cellulose (HPC), hydroxypropyl methylcelluclose (HPMC), carboxymethyl cellulose (CMC), gelatin, whey, and nanocellulose.

In some embodiments, mixing the RuBisCO and the one or more solvents to form a slurry may further comprise agitating for about two minutes and blending for about 10 minutes. In other embodiments, drying the slurry in the mold may further comprise allowing the slurry to dry overnight at room temperature. In still other embodiments, drying the slurry in the mold may further comprise placing the slurry in the mold in a forced air oven at about 70 degrees C. to about 80 degrees C. for about 10 minutes to about 20 minutes. In some embodiments, the RuBisCO protein films formed may have a moisture content of about 10% to about 12%. In some embodiments, the RuBisCO protein film(s) formed may be edible.

In another aspect, a method of producing RuBisCO protein films may include the steps of: extracting RuBisCO protein from tobacco; purifying the obtained RuBisCO protein; combining the RuBisCO protein with one or more solvents, where the one or more solvents may be about 10% w/v the RuBisCO protein: mixing the RuBisCO and the one or more solvents to form a slurry, where the mixing further comprises agitating and blending; heating the slurry to about 70 degrees C. and holding the slurry at about 70 degrees C. while stirring for about 30 minutes; cooling the slurry to at least about 45 degrees C.; dispensing the slurry into one or more molds for film formation: drying the slurry in the one more molds; and removing the one or more RuBisCO protein films formed within the one or more molds.

In some embodiments, the one or more solvents may be selected from a group consisting of: water, ethanol, glycerol, propylene glycol, polypropylene glycol, hexane, a citrate solution, a phosphate solution, a chloride solution, a sodium sulfate solution, a potassium sulfate solution, a sodium hydroxide solution, a potassium hydroxide solution, a calcium hydroxide solution, a magnesium hydroxide solution, a hydrochloric acid solution, a phosphoric acid solution, a citric acid solution, a sodium carbonate solution, and a potassium carbonate solution.

In some embodiments, the method may further comprise adding one or more additional additives selected from a group consisting of: one or more crosslinking agents, one or more plasticizers, and one or more reinforcers. In some embodiments, the crosslinking agent(s) may be selected from a group consisting of glutaraldehyde, glyoxal, and formaldehyde. In other embodiments, the plasticizer(s) may be selected from a group consisting of monosaccharides, disaccharides, oligosaccharides, polyols, and lipids. In still other embodiments, reinforcers are selected from a group consisting of sodium alginate, pectin, carrageenan, gellan, agar, gum acacia or gum Arabic, tragacanth, karaya, guar, locust bean, pullulan, xanthan, hydroxypropyl cellulose (HPC), hydroxypropyl methylcelluclose (HPMC), carboxymethyl cellulose (CMC), gelatin, whey, and nanocellulose.

In still another aspect, a method of producing one or more RuBisCO protein films, the method may include: extracting RuBisCO protein from tobacco; purifying the obtained RuBisCO protein; combining the RuBisCO protein with one or more solvents, where the one or more solvents may be about 10% w/v the RuBisCO protein; mixing the RuBisCO and the one or more solvents to form a slurry; adding one or more additional additives selected from a group consisting of: one or more crossing-linking agents, one or more plasticizers, and one or more reinforcers; heating the slurry to about 70 degrees C. and holding the slurry at about 70 degrees C. while stirring for about 30 minutes; cooling the slurry to at least about 45 degrees C.; dispensing the slurry into one or more molds for film formation; drying the slurry in the one more molds; and removing the one or more RuBisCO protein films formed within the one or more molds, where the one or more RuBisCO protein films have a moisture content of about 10% to about 12%.

### BRIEF DESCRIPTION OF THE DRAWINGS

FIG. 1 is a photographic illustration of the films resulting from Examples 2 through 5 described herein.

FIG. 2 illustrates an exemplary method of producing RuBisCO films.

### DETAILED DESCRIPTION

Generally, the present invention provides methods for generating protein-based films utilizing ribulose-1,5-bisphosphate carboxylase-oxygenase (hereinafter “RuBisCO”), whose total molecular weight is about 550 kD. Furthermore, when subjected to heating and other processing, particularly in an aqueous slurry, RuBisCO is known to exhibit various functional properties that may be desirable in a protein source for film formation. Such properties include solubility, viscosity builder, gel formation, water retention, foaming, emulsifying attributes, and the like. As discussed previously, RuBisCO is considered the most abundant plant protein known, as it is present in every plant that undergoes photosynthesis. RuBisCO may comprise up to about 25% of the total protein content of a leaf and up to about 10% of the solid matter of a leaf. In particular, in one embodiment, the RuBisCO proteins utilized in the formation of such films may be extracted from one or more plants of the Nicotiana species (generally referred to herein as “tobacco”), which may have the highest potential yield per acre of RuBisCO of all plants. Furthermore, the use of plant protein in the production of protein films, in particular the use of a non-food plant source (for example tobacco), may be a more sustainable and more environmentally friendly source as compared to other protein sources including, for example proteins sourced from animals.

Although the present disclosure focuses primarily on RuBisCO protein extracted from a plant of the Nicotiana species, it is noted that various methods disclosed herein may be applicable to RuBisCO extracted from sources other than tobacco plants. In some embodiments, RuBisCO proteins may be extracted from any photosynthesizing plant. In other embodiments, RuBisCO proteins may be extracted from other photosynthesizing organisms, including, but not limited, to various species of photosynthetic bacteria.

The plant of the Nicotiana species may be employed in either an immature or mature form, and may be used in either a green form or a cured form, as described in U.S. Pat. Pub. No. 2012/0192880 to Dube et al., which is incorporated by reference herein. The tobacco material may be subjected to various treatment processes such as, refrigeration, freezing, drying (e.g., freeze-drying or spray-drying), irradiation, yellowing, heating, cooking (e.g., roasting, frying, or boiling), fermentation, bleaching, or otherwise subjected to storage or treatment for later use. In some embodiments, harvested tobacco can be sprayed with a buffer or antioxidant (e.g., a sodium met-abisulfite buffer) to prevent the green plants from browning prior to extract and purification treatments. Other exemplary processing techniques are described, for example, in U.S. Pat. Pub. Nos. 2009/0025739 to Brinkley et al. and 2011/0174323 to Coleman, III et al., which are incorporated by reference herein. Additionally, at least a portion of the plant of the Nicotiana species may be treated with enzymes and/or probiotics before or after harvest, as discussed in U.S. Pat. Pub. No. 2013/0269719 to Marshall et al. and U.S. Pat. No. 9,485,953 to Moldoveanu, which are incorporated herein by reference.

Generally, any method known in the art may be used for the extraction of RuBisCO. Including, but not limited to those methods described in U.S. Pat. No. 9,301,544 to Mua et al., U.S. Pat. No. 9,175,052 to Gerardi et al., U.S. Pat. Pub. No. 2016/0192697 to Mua et al., and U.S. Pat. Pub. No. 2016/0029663 to Gerardi et al., all of which are incorporated by reference herein in their entireties. Other exemplary methods for extracting proteins, such as RuBisCO, from tobacco and other plants include, but are not limited to, those described in U.S. Pat. No. 7,337,782 to Thompson; U.S. Pat. No. 6,033,895 to Garger et al.; U.S. Pat. No. 4,941,484 to Clapp et al.; U.S. Pat. Nos. 4,588,691 and 4,400,471 to Johal; U.S. Pat. No. 4,347,324 to Kwanyuen et al., U.S. Pat. No. 4,340,676 to Bourque; U.S. Pat. No. 4,333,871 to DeJong; U.S. Pat. Nos. 4,289,147 and 4,268,632 to Wildman et al.; U.S. Pat. Nos. 3,959,246, 3,823,128, and 3,684,520 to Bick-off et al.; U.S. Pat. Pub. Nos. 2010/0093054 to Lo et al. and 2013/0072661 to Kale; U.S. Pat. Pub. 2014/0271952 to Mua et al.; Int'l Appl. Publ. Nos. WO2011/078671 to Van de Velde et al. and WO2008/143914 to Lo; and EP Pat. Publ. Nos. EP 2403888 to Parker et al.; EP 1691759 to Boddupalli et al.; and EP 1067946 to Brinkhaus et al., which are all incorporated by reference herein in their entireties.

Generally, an example embodiment of the extraction process includes creating what is commonly referred to in the industry as “green juice” by extracting a whole plant, for example a tobacco plant, with a buffer solution. This “green juice” may be subjected to centrifugation in order to remove debris. Supernatant collected from this centrifugation may then be filtered. First, tangential flow filtration with a filter size of about 0.1 microns may be used to collect a first fraction containing RuBisCO. This fraction, F1, is composed primarily of the largest RuBisCO proteins (which may range from about 80 kD to about 700 kD). A second filtration system with a filter size of about 10 kD may then be used to collect a second fraction, for example the F2 protein fraction, which is a mixture of smaller soluble proteins of cytoplasmic and chloroplastic origin. F2 proteins and peptides generally have molecular weights ranging from about 3 kD to about 100 kD, but this fraction may also contain minor amounts of the larger species (500-600 kD) that make it through the 0.1 micron filter. This general process may result in the production of pellets or other non-liquid product (e.g. powder) that contain various starches and proteins, including RuBisCO, and a liquid extract and distillate that may contain nicotine. These pellets and other non-liquid products (e.g. powder) may be used in the production of films, and/or they may be used in other downstream processing. For example, in some embodiments, the RuBiSCO may be extracted from a plurality of tobacco leaves and spray-dried into a powder. In some embodiments, this resulting powder may be brown in color, aroma-free, and/or tasteless. In other embodiments, the liquid extract and distillate resulting from this process may be discarded.

In some embodiments, the extracted RuBisCO proteins may be, optionally, further processed in order to improve various qualities of the protein sample, including for example purity. In some embodiments, the RuBisCO utilized in film production may be about 70% to about 80% pure. In other embodiments, the extracted RuBisCO may undergo further processing in order to concentrate the extracted proteins. In some embodiments, further processing may include adjusting the pH, heating and/or stirring of RuBisCO slurry, retentate, or concentrate so as to re-solubilize the protein. In other embodiments, the concentrate may be also be filtered. In still other embodiments, the RuBisCO retentate may be sprayed or freeze dried into a powder.

Additionally, the extracted RuBisCO proteins may also be, optionally, combined with other proteins, which may occur either before or after the additional processing previously described. In some instances these other proteins may be derived from the same plant source as the RuBisCO, for example proteins contained in the F2 faction. In other instances the other protein may be derived from a separate plant source, an animal source, or any other source of proteins known in the art. Some non-limiting examples of other proteins that may be combined with the extracted RuBisCO protein include collagen, corn zein, wheat gluten, soy, casein, mung bean, whey, gelatin, and/or pea proteins.

Protein-based films are generally created from solutions containing the protein and a solvent, carrier, or the like. In some embodiments, the solvent or carrier may be water, ethanol, or a combination thereof. As the solvent, carrier, or the like evaporates a film may be formed. In order to form the chain-to-chain interaction and corresponding structures required for the formation of protein films, it may be required to denature the proteins. In some embodiments elevated temperatures may be used to denature proteins. In other embodiments, elevated temperatures may be combined with the use of pressure to denature proteins. In still other embodiments, acid, bases, and/or various solvents may be used to denature the proteins. In some embodiments, enzymes and/or buffers, may be used to denature proteins. Generally, any means of denaturing or hydrolyzing proteins to enable molecular structure bonding and/or the realignment of molecular structures known in the art may be used. Once denatured, the extended protein chains may associate through various types of bonding, including, but not limited to, hydrogen bonds, ionic bonds, hydrophobic bonds, and/or covalent bonds. The amount and types of bonding may be affected by the degree of denaturation of the protein, as well as by the amino acid composition and/or concentration of the protein.

Increased interactions, bonding, and/or crosslinking may result in films that are stiffer and less permeable to gases, vapors, or liquids. Furthermore, various chemical, physical, and/or enzymatic treatments and/or modifications may be applied to the proteins with or without additional materials (e.g. additional polymers) in order to improve certain qualities of the resulting protein films including, but not limited to film strength. Various chemical treatments (e.g. acids, alkali solutions, and/or crosslinking agents) may increase the desirable properties of the resulting protein film. In some embodiments, for example, sodium and potassium salts and their buffers may be used to enable RuBisCO film formation. In other embodiments, acids (e.g. hydrochloric acid, phosphoric acid, citric acid, acetic acid, etc.) may be used to enable RuBisCO film formation. In still other embodiments, heat of up to 121 degrees C. and pressure of up to 21 psi may be used to enable RuBisCO film formation. In some embodiments, protease/proteolytic and peptidase enzymes may be used to enable RuBisCO film formation.

Some non-limiting examples of chemicals that may be used as covalent cross linking agents include aldehydes such as glutaraldehyde, glyoxal and/or formaldehyde. In some embodiments, formaldehyde may be a desirable cross-linking agent. In other embodiments, particularly where the film may be edible, formaldehyde may not be desirable as a cross-linking agent.

Various factors may affect the formation of, and properties of, protein films. These factors include, but are not limited to: the source of the protein or type of material, in particular whether the protein is hydrophobic or hydrophilic; the structure of the polymer; pH; the drying temperature of the protein film while being cast; protein concentration in the film-preparation solution; relative humidity; and, whether any, or what kinds of additives (e. g. plasticizers), may be included in the protein film solution and production process. For example, crosslinking agents may facilitate inter-chain interaction, while plasticizers may allow the film to be more flexible.

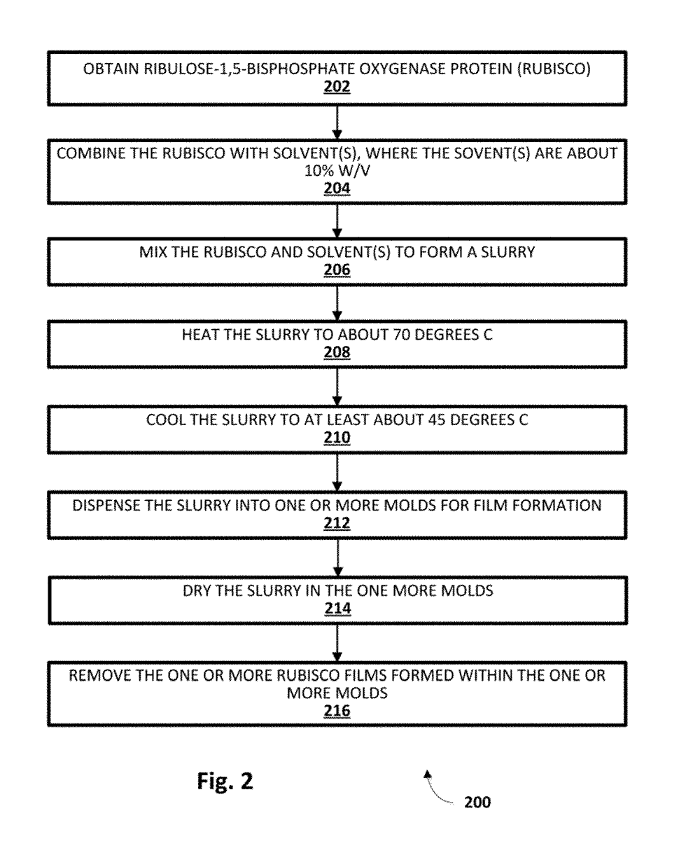

An exemplary embodiment of forming RuBisCO protein films is disclosed herein and illustrated in FIG. 2. This exemplary embodiment requires obtaining RuBisCO, block 202. This RuBisCO may be extracted from tobacco, as described previously, or may be obtained from other sources, such as other plants or photosynthetic organisms. In some embodiments, the RuBisCO may be in a dry form, for example, as a powder form. The RuBisCO may be added to a solvent, block 204. In some embodiments, the solvent may be water. In other embodiments, the solvent may be ethanol, glycerol, propylene glycol, polypropylene glycol, or hexane. In still other embodiments, the solvent may be a solution, for example an acid solution (for example, hydrochloric acid, phosphoric acid, citric acid, or the like), a base solution (for example, sodium hydroxide, potassium hydroxide, calcium hydroxide, magnesium hydroxide, or the like), a salt solution (for example, citrate, phosphate, chloride, sodium sulfate, potassium sulfate, or the like), or a buffer solution (for example sodium carbonate, potassium carbonate, or the like).

The amount of RuBisCO added may be dependent on the amount of solvent. In some embodiments the amount of RuBisCO added may be about 10% w/v: as an illustrative example, in such an embodiment, the amount of RuBisCO may be 80 g when it is added to 800 mL of water.

The RuBisCO and the solvent may be mixed to form a slurry, block 206. In some embodiments, this mixing may be with an agitating mixer. In other embodiments, this mixing may be with a blending mixer, for example a Waring® blender. In still other embodiments, this mixing may be with a combination of mixing techniques, such as agitation, blending, or any other mixing technique known in the art. As an illustrative example, in some embodiments the RuBisCO and solvent (e.g. water) may be mixed at a medium speed for about two minutes with an agitating mixer and then transferred to a blender and blended for about 10 minutes at a medium speed (e.g. about 45 rpm). This mixing may result in the solvent-RuBisCO mixture forming into a slurry.

The solvent-RuBisCO slurry may be heated, block 208. Heat may be applied through a variety of mechanisms, including through the use of both direct and indirect heat sources. For example, the slurry may be heated using a flame, hot plate, oven or the like. In some embodiments, the slurry mixture may be heated to about 70 degrees C. In other embodiments, the slurry mixture may be heated to about 70 degrees C. and held at 70 degrees for about 30 minutes. In still other embodiments, the RuBisCO slurry mixture may be heated to about 70 degrees C. and held at 70 degrees for about 30 minutes while stirred. In some embodiments, the slurry mixture may be periodically stirred, while in other embodiments the slurry mixture may be constantly stirred.

The heated slurry may be allowed to cool until it reaches less than about 45 degrees C., block 210. In some embodiments, a plasticizer may be added to the slurry. Generally, plasticizers are low molecular weight, non-volatile compounds used as additives or incorporated into other material in order to increase flexibility, workability, and dispensability. The process of plasticizing a protein-based polymer may be affected by the selected plasticizer's molecular weight, as well as the number and position of various hydroxyl groups. Other properties that may be affected by the addition of a plasticizer include, but are not limited to, crystallinity, optical clarity, electric conductivity, and ability to resist degradation. One or more of various plasticizers known in the art may be selected for use in protein films, including, but not limited to, monosaccharides, disaccharides and/or oligosaccharides (e.g. glucose syrups or glucose fructose honey), polyols (e.g. glycerol and derivatives, polyethylene glycols, and sorbitol) and lipids and derivatives (e.g. fatty acids, monoglycerides and their esters, acetoglycerides, phospholipids, and other emulsifiers). The chemical composition of the selected plasticizer, including the configuration of functional groups, may affect the way the plasticizer(s) interact with the polymer.

In some embodiments, an additional substance may be added to the RuBisCO slurry to strengthen and reinforce the chemical structure of the resulting film. In some embodiments, such a substance (e.g. a “reinforcer”) may be sodium alginate, while in other embodiments it may be pectin. In still other embodiments, the reinforcer may include, but is not limited to: carrageenan, gellan, agar, gum acacia or gum Arabic, tragacanth, karaya, guar, locust bean, pullulan, xanthan, hydroxypropyl cellulose (HPC), hydroxypropyl methylcelluclose (HPMC), carboxymethyl cellulose (CMC), gelatin, and/or whey. In still other embodiments, nanocellulose may be used as a reinforcer, as nanocelluse may act as both a thickening agent and as a film reinforcing agent.

In some embodiments, one or more plasticizers may be added to the RuBisCO slurry. In some embodiments, one or more reinforcers may be added to the RuBisCO slurry. In still other embodiments, a combination of one or more plasticizers and one or more reinforcers may be added to the RuBisCO slurry. For example, in some embodiments, glycerin may be added to the slurry as a plasticizer and/or sodium alginate added as a reinforcer.

The RuBisCO slurry, with or without the addition of one or more plasticizers and/or one or more reinforcers, is portioned out into aliquots. The size of the aliquot may depend on the weight and/or volume of the desired size of the resulting film, for example the larger the desired film the larger the aliquot of RuBisCO slurry. In some embodiments, the slurry may be portioned into 100 g aliquots for film formation.

The RuBisCO slurry, with or without the addition of one or more plasticizers and/or one or more reinforcers, may be formed into RuBisCO films. The film formation may occur through the placement of the slurry into one or more molds in order to produce a desired shaped film, block 212. The thickness, shape, etc. of the resulting RuBisCO film may depend on the mold used for its formation and/or the way the RuBisCO slurry was placed or poured into the mold. In some embodiments, the RuBisCO slurry may be placed on one or more stainless steel plates (molds) to form one or more thin RuBisCO films. In other embodiments, a film casting knife may be used to place the RuBisCO slurry into one or more stainless steel plates. In some embodiments, the one or more resulting RuBisCO films may be about 0.2 μm to about 1.0 μm thick.

The RuBisCO slurry is allowed to dry in the mold, block 214. In some embodiments, the RuBisCO slurry is allowed to air dry at room temperature, for example by leaving it in the mold overnight. In other embodiments, the RuBisCO slurry in the mold may be dried using heat, including using both direct and indirect heat sources. In some embodiments, the RuBisCO film may be dried in a forced air oven at about 70 degrees C. to about 80 degrees C. for about 10 to about 20 minutes. In other embodiments, the RuBisCO films may be dried until the moisture content of the film is about 10% to about 12%. The dried RuBisCO films may be removed from the mold, block 216.

Films resulting from the processes described herein may be suitable for various biomedical uses, various packaging applications (e.g. food packaging), or the like. In some embodiments, for example, resulting RuBisCO films may be used in breath freshening strips or energy strips that may be (typically) placed on/under the tongue to dissolve. In other embodiments, the resulting RuBisCO films may be used in feminine products. Some non-limiting examples of biomedical uses may include incorporation of the films into wound dressings, use as a film coating on biomedical equipment, skin patches or oral strips for pharmaceutical delivery, or the like.

The resulting RuBisCO films may be used in a variety of packaging applications, including for use with both food and non-food products. In some embodiments, the RuBisCO film may be edible. For example, in some embodiments the resulting RuBisCO film may be used to encapsulate food or pharmaceutical products for human consumption. In other embodiments, the RuBisCO film may be used as a casing for food products, including, but not limited to, packaging for tobacco products (e.g. snus). In still other embodiments, the RuBisCO film may be incorporated into a wrap, pouch, or bag, which may or may not be used in conjunction with food products. Food packaging may have more specific requirements with respect to moisture or oxygen permeability. For example, food packaging may need to function as a barrier to the exchange of moisture and/or oxygen. A moisture barrier may be added to a RuBisCO film through and atomic layer deposition method, for example the method described in U.S. Pat. Pub. No. 2016/0135499 to Sebastian. et al. Additionally, the addition of nanocellulose during film production may also reduce oxygen transmission rate.

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either.” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example. “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one. A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03. It should be understood that certain expressions and reference signs used in the claims pursuant to Rule 6.2(b) of the Patent Cooperation Treaty (“PCT”) do not limit the scope.

### EXAMPLES

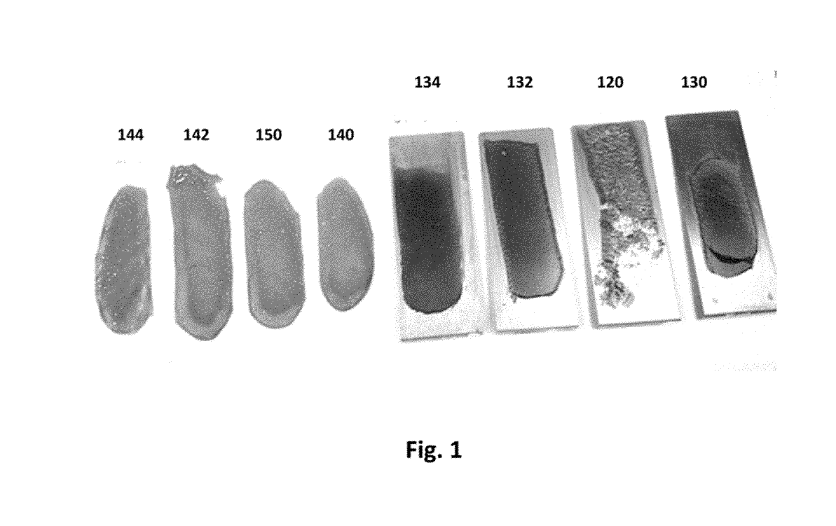

The above described methods may be used for the formation of a variety of RuBisCO films. The following examples represent various RuBisCO films formed. The results of Examples 2 through 5 are shown in Table 1 and FIG. 1. Table 1 provides a description of various properties of the resulting RuBisCO films, including: the consistency of the film, such as whether the protein film simply coated the stainless steel cast and/or whether the protein film was able to be peeled off of the stainless steel cast and yielded a free standing film: the flexibility of the resulting protein films; and, the color of the resulting films.

### Example 1

A graduated cylinder was used to measure 800 mL of water, which was placed into a beaker. A total of 80 g (10% w/v) of RuBisCO powder extracted from a tobacco plant was measured and added to the water in the beaker. The RuBisCO and water were mixed for approximately two minutes using an agitator mixer on a medium speed setting. Following this two minutes of mixing the mixture was transferred to a Waring® blender mixer and blended for approximately 10 minutes at a medium speed (e.g. about 45 rpm) resulting in a RuBisCO slurry. The RuBisCO slurry was then heated to and held at 70 degrees C. on a hot plate with consistent stirring for approximately 30 minutes. After heating and stirring, the slurry was cooled to at least 45 degrees C. and weighed out into 100 g aliquots for film formation.

### Example 2

One aliquot from Example 1 was placed onto a stainless steel cast and then formed into a thin film (approximately 0.2 μm to 1.0 μm) using a laboratory draw down cast film knife. The resulting film was allowed to dry overnight at room temperature. After drying, the consistency, flexibility, and color of the film were each noted. The results of Example 2 are shown in Table 1 below and FIG. 1 as 120.

### Example 3

Three grams of glycerin were measured and added to each of three aliquots from Example 1. Each of these three aliquots were placed onto a stainless steel casts and formed into thin films (approximately 0.2 μm to 1.0 μm) using a laboratory draw down cast film knife. The resulting films were allowed to dry overnight at room temperature. After drying, the consistency, flexibility, and color of each film were each noted. The results of Example 3 are shown in Table 1 below as Example 3A, 3B, and 3C and in FIG. 1 as 130, 132, and 134 respectively.

### Example 4

Three grams of glycerin and 2 g of sodium alginate (Protanal® 1815) were measured and added to each of three aliquots from Example 1. Each of these three aliquots were placed onto a stainless steel casts and formed into thin films (approximately 0.2 μm to 1.0 μm) using a laboratory draw down cast film knife. The resulting films were allowed to dry overnight at room temperature. After drying, the consistency, flexibility, and color of each film were each noted. The results of Example 4 are shown in Table 1 below as Example 4A, 4B, and 4C and in FIG. 1 as 140, 142, and 144 respectively.

### Example 5

Two grams of glycerin and 2 g of sodium alginate (Protanalt 1815) were measured and added to an aliquot from Example 1. This aliquot was placed onto a stainless steel cast and formed into a thin film (approximately 0.2 μm to 1.0 μm) using a laboratory draw down cast film knife. The resulting film was allowed to dry overnight at room temperature. After drying the consistency, flexibility, and color of the film were each noted. The results of Example 5 are shown in Table 1 below and in FIG. 1 as 150.

TABLE 1
Results from Examples 2-5.

| | Ex. 2 | Ex. 3A | Ex. 3B | Ex. 3C | Ex. 4A | Ex. 4B | Ex. 4C | Ex. 5 |
|---|---|---|---|---|---|---|---|---|
| Protein (g) | 10 | 10 | 10 | 10 | 10 | 10 | 1.0 | 1.0 |
| Water (g) | 100 | 100 | 100 | 100 | 100 | 100 | 1.00 | 1.00 |
| Glycerin (g) | 0 | 3 | 3 | 3 | 3 | 3 | 3 | 2 |
| Sodium | 0 | 0 | 0 | 0 | 2 | 2 | 2 | 2 |
| Alginate (g) | | | | | | | | |
| Film | Coating | Coating | Coating | Coating | Free | Free | Free | Free |
| Consistency | | | | | Standing | Standing | Standing | Standing |
| Film | Brittle | Hard | Hard | Hard | Flexible | Flexible | Flexible | Flexible |
| Flexibility | | | | | | | | |
| Film Color | Tan/ | Tan/ | Tan/ | Tan/ | Tan/ | Tan/ | Tan/ | Tan/ |
| | Brown | Brown | Brown | Brown | Brown | Brown | Brown | Brown |

What is claimed is:

1. A method of producing one or more ribulose-1,5-bisphosphate oxygenase protein films, the method comprising:
obtaining ribulose-1,5-bisphosphate oxygenase protein;
combining the ribulose-1,5-bisphosphate oxygenase protein with one or more solvents, wherein the one or more solvents are about 10% w/v the ribulose-1,5-bisphosphate oxygenase protein;
mixing the ribulose-1,5-bisphosphate oxygenase and the one or more solvents to form a slurry;
heating the slurry to about 70 degrees C.;
cooling the slurry to at least about 45 degrees C.;
dispensing the slurry into one or more molds for film formation;
drying the slurry in the one more molds; and
removing the one or more ribulose-1,5-bisphosphate oxygenase protein films formed within the one or more molds.

2. The method of claim 1, wherein the ribulose-1,5-bisphosphate oxygenase protein is obtained from one or more tobacco plants.

3. The method of claim 1, wherein the method further comprises purifying the obtained ribulose-1,5-bisphosphate oxygenase protein.

4. The method of claim 1, wherein the one or more solvent solutions are selected from a group consisting of: water, ethanol, glycerol, propylene glycol, polypropylene glycol, hexane, a citrate solution, a phosphate solution, a chloride solution, a sodium sulfate solution, a potassium sulfate solution, a sodium hydroxide solution, a potassium hydroxide solution, a calcium hydroxide solution, a magnesium hydroxide solution, a hydrochloric acid solution, a phosphoric acid solution, a citric acid solution, a sodium carbonate solution, and a potassium carbonate solution.

5. The method of claim 1, wherein the method further comprising adding one or more additional additives selected from a group consisting of: one or more crosslinking agents, one or more plasticizers, and one or more reinforcers.

6. The method of claim 5, wherein the one or more crosslinking agents are selected from a group consisting of glutaraldehyde, glyoxal, and formaldehyde.

7. The method of claim 5, wherein the one or more plasticizers are selected from a group consisting of monosaccharides, disaccharides, oligosaccharides, polyols, and lipids.

8. The method of claim 5, wherein the one or more reinforcers are selected from a group consisting of sodium alginate, pectin, carrageenan, gellan, agar, gum acacia or gum Arabic, tragacanth, karaya, guar, locust bean, pullulan, xanthan, hydroxypropyl cellulose, hydroxypropyl methylcelluclose (HPMC), carboxymethyl cellulose (CMC), gelatin, whey, and nanocellulose.

9. The method of claim 1, wherein mixing the ribulose-1,5-bisphosphate oxygenase and the one or more solvents to form a slurry further comprises:
agitating for about 2 minutes; and
blending for about 10 minutes.

10. The method of claim 1, wherein drying the slurry in the mold further comprises allowing the slurry to dry overnight at room temperature.

11. The method of claim 1, wherein drying the slurry in the mold further comprises placing the slurry in the mold in a forced air oven at about 70 degrees C. to about 80 degrees C. for about 10 minutes to about 20 minutes.

12. The method of claim 1, wherein the one or more ribulose-1,5-bisphosphate oxygenase protein films formed have a moisture content of about 10% to about 12%.

13. The method of claim 1, wherein the one or more ribulose-1,5-bisphosphate oxygenase protein films formed are edible.

14. A method of producing one or more ribulose-1,5-bisphosphate oxygenase protein films, the method comprising:
extracting ribulose-1,5-bisphosphate oxygenase protein from tobacco;
purifying the exacted ribulose-1,5-bisphosphate oxygenase protein;
combining the purified ribulose-1,5-bisphosphate oxygenase protein with one or more solvents, wherein the one or more solvents is about 10% w/v the ribulose-1,5-bisphosphate oxygenase protein;
mixing the ribulose-1,5-bisphosphate oxygenase and the one or more solvents to form a slurry, wherein the mixing further comprises agitating and blending;
heating the slurry to about 70 degrees C. and holding the slurry at about 70 degrees C. while stirring for about 30 minutes;
cooling the slurry to at least about 45 degrees C.;
dispensing the slurry into one or more molds for film formation;
drying the slurry in the one more molds; and
removing the one or more ribulose-1,5-bisphosphate oxygenase protein films formed within the one or more molds.

15. The method of claim 14, wherein the one or more solvents are selected from a group consisting of: water, ethanol, glycerol, propylene glycol, polypropylene glycol, hexane, a citrate solution, a phosphate solution, a chloride solution, a sodium sulfate solution, a potassium sulfate solution, a sodium hydroxide solution, a potassium hydroxide solution, a calcium hydroxide solution, a magnesium hydroxide solution, a hydrochloric acid solution, a phosphoric acid solution, a citric acid solution, a sodium carbonate solution, and a potassium carbonate solution.

16. The method of claim 14, wherein the method further comprising adding one or more additional additives selected from a group consisting of: one or more crosslinking agents, one or more plasticizers, and one or more reinforcers.

17. The method of claim 16, wherein the one or more crosslinking agents are selected from a group consisting of glutaraldehyde, glyoxal, and formaldehyde.

18. The method of claim 16, wherein the one or more plasticizers are selected from a group consisting of monosaccharides, disaccharides, oligosaccharides, polyols, and lipids.

19. The method of claim 16, wherein the one or more reinforcers are selected from a group consisting of sodium alginate, pectin carrageenan, gellan, agar, gum acacia or gum Arabic, tragacanth, karaya, guar, locust bean, pullulan, xanthan, hydroxypropyl cellulose, hydroxypropyl methylcelluclose (HPMC), carboxymethyl cellulose (CMC), gelatin, whey, and nanocellulose.

20. A method of producing one or more ribulose-1,5-bisphosphate oxygenase protein films, the method comprising:
extracting ribulose-1,5-bisphosphate oxygenase protein from tobacco;
purifying the extracted ribulose-1,5-bisphosphate oxygenase protein;
combining the purified ribulose-1,5-bisphosphate oxygenase protein with one or more solvents, wherein the one or more solvents is about 10% w/v the ribulose-1,5-bisphosphate oxygenase protein;
mixing the ribulose-1,5-bisphosphate oxygenase and the one or more solvents to form a slurry;
adding one or more additional additives selected from a group consisting of: one or more crossing-linking agents, one or more plasticizers, and one or more reinforcers;
heating the slurry to about 70 degrees C. and holding the slurry at about 70 degrees C. while stirring for about 30 minutes;
cooling the slurry to at least about 45 degrees C.;
dispensing the slurry into one or more molds for film formation;
drying the slurry in the one more molds; and
removing the one or more ribulose-1,5-bisphosphate oxygenase protein films formed within the one or more molds, wherein the one or more ribulose-1,5-bisphosphate oxygenase protein films have a moisture content of about 10% to about 12%.
